(19) United States
(12) Patent Application Publication
(10) Pub. No.: US 2024/0410897 A1
(43) Pub. Date: Dec. 12, 2024

(54) INTACT MASS SPECTROMETRY FOR DIRECT PROTEIN FOOTPRINTING DOSIMETRY USING COVALENT LABELING

(71) Applicant: Case Western Reserve University, Cleveland, OH (US)

(72) Inventors: Mark R. Chance, Moreland Hills, OH (US); Erik Farquhar, Shirley, NY (US); Janna Kiselar, Cleveland, OH (US)

(21) Appl. No.: 18/680,965

(22) Filed: May 31, 2024

Related U.S. Application Data
(60) Provisional application No. 63/506,745, filed on Jun. 7, 2023.

(51) Int. Cl. G01N 33/68

(52) U.S. Cl. CPC G01N 33/6848; G01N 2458/15

(57) ABSTRACT

The present disclosure provides a protein footprinting method based on mass spectrometry of an intact protein labeled with covalent labeling reagents, including hydroxyl radicals. The method involves intact MS screening of labeled samples and measurement of the extent of observed labeling (e.g., average oxidation events per protein) from the intact mass spectra. Advantageously, this direct, intact mass spectrometry-based approach can be used to determine a suitable dose range of the labeling agent relative to a subject protein in order to produce adequately labeled protein while avoiding damage to the subject protein caused by excessive use of the labeling agent.

### CROSS-REFERENCE TO RELATED APPLICATIONS

This application claims priority to U.S. Provisional patent application No. 63/506,745, filed on Jun. 7, 2023, the content of which is hereby incorporated by reference in its entirety.

### STATEMENT REGARDING FEDERALLY FUNDED RESEARCH

This invention was made with government support under GM141078 awarded by the National Institutes of Health. The government has certain rights in the invention.

### BACKGROUND

Protein footprinting is a structural biology method to evaluate the structure of proteins in their native solution state, via treatment of the biomolecule with a controlled dose of chemical labels that react with regions of the protein that are exposed to aqueous solvent. The dose required is contingent upon the protein of interest and other components of the solution that can react with labeling agent. This dose needs to be appropriately poised so that there is adequate labeling of the regions of interest, but not so much that the sample state is perturbed. If the samples are changed or destroyed by damage from the label, data may be corrupted by this over labeling. Further if samples are under labelled, structural coverage and data quality are reduced. Consequently, methods to accurately assess footprinting dose are key to successfully applying protein footprinting to research problems.

Synchrotron X-ray footprinting is a well-validated method to assess protein structure in the native solution state, via X-ray radiolysis of water to generate reactive hydroxyl radicals that react with solvent accessible side chains of proteins. In this method the optimization of hydroxyl radical dose is typically performed using an indirect Alexa488 fluorescence assay, but evaluation of the experiment outcome relies upon bottom-up mass spectrometry (MS) measurements to determine sites and extent of oxidative labeling, which occurs well after exposure of samples at the synchrotron. It remains a challenge to provide adequate labeling to ensure detection of labeled products, while avoiding excessive doses that cause the readout to reflect effects of labeling. A direct evaluation of the extent of labeling to provide guidance on dose and “safe” dose ranges would provide immediate feedback on experimental outcomes prior to embarking on detailed sample analyses.

Unfortunately, all current dosimetry methods are indirect, they sample the solution conditions of the labeling reaction through use of optical or fluorescence methods and such labeling problems are not revealed until after downstream bottom-up analysis after protease digestion. This increases the time for optimizing and thus completing a successful experiment.

Thus, there remains a need for methods suitable for directly and effectively evaluating the extent of protein labeling to provide guidance on dose of chemical labels, which can be useful as immediate feedback on experimental outcomes prior to embarking on detailed sample analyses.

### SUMMARY

The present disclosure generally relates to methods for direct assessment and control of the extent of protein labeling for protein footprinting using covalent labeling reagents including hydroxyl radical.

In one aspect, the present disclosure provides a method of analyzing protein structure. The method can comprise: (a) labeling a protein with a label to generate an intact labeled protein, wherein the label comprises a hydroxyl radical; (b) obtaining a mass spectrum of the intact labeled protein; and (c) analyzing the mass spectrum of the intact labeled protein. In some embodiments, analyzing the mass spectrum of the intact labeled protein comprising determining an average number of labels per protein molecule. In some embodiments, the method further comprises (d) determining a dose of the label based on the average number of labels per protein molecule. In some embodiments, the method further comprises (e) repeating steps (a)-(d), in which the protein is labeled with the label according to the dose of the label.

In another aspect, the present disclosure provides a method of determining a dose of hydroxyl radicals for protein labeling. The method can comprise: (i) labeling a protein with hydroxyl radicals to generate an intact labeled protein; (ii) obtaining a mass spectrum of the intact labeled protein; (iii) analyzing the mass spectrum of the intact labeled protein to determine the dose of hydroxyl radicals. The method can further comprise (iv) repeating (i)-(iii) to obtain a range of doses of hydroxyl radicals.

In another aspect, the present disclosure provides method of labeling a protein. The method can comprise: labeling a protein with a label to generate an intact labeled protein, wherein the label comprises a hydroxyl radical; subjecting the intact labeled protein to mass spectrometry, thereby determining a dose of the label relative to the protein; and further labeling the protein with the label according to the dose of the label.

In another aspect, the present disclosure provides a method of performing protein footprinting. The method can comprise: labeling a protein according to the method of labeling a protein as described herein to generate a labeled protein sample; and analyzing the labeled protein sample, thereby a structural character of the protein is determined.

In some embodiments, the protein as described herein is denatured prior to mass spectrometry. The protein can be, for example, at least 50 amino acids in length. The label as described herein can be a combination of a hydroxyl radical and at least one different radical. In some embodiments, the label is a hydroxyl radical. The label or labeling reagents as described herein can be generated, for example, by X-ray radiolysis.

In yet another aspect, the present disclosure provides an intact labeled protein produced by the protein labeling method as described herein.

### BRIEF DESCRIPTIONS OF THE DRAWINGS

FIG. 1 shows representative Alexa488 OH dose-response rates for a range of aluminum attenuator thicknesses for 20 μM lysozyme (●) and 20 μM lysozyme+80 μM GDP (▪) The dashed lines depict the empirical 25-80 s⁻¹ Alexa OH dose-response rate window for optimal labeling.

FIG. 2 shows representative intact mass spectra under denaturing conditions obtained for 20 μM lysozyme exposed to X-rays for 0, 12, 20, and 30 milliseconds with 864 μm aluminum attenuation, with charge states indicated above each peak (left) and expanded views of the +10 charge state, showing new signals developing with increased exposure time (right).

FIG. 3 shows representative deconvolved mass spectra for 20 μM lysozyme (panel a) and 20 μM lysozyme+80 μM GDP (panel b) at 864 μm aluminum attenuation, 20 μM lysozyme (panel c) and 20 μM lysozyme+80 μM GDP (panel d) at 508 μm aluminum attenuation. Curves from bottom to top are results of exposures at 0 ms, 12 ms, 20 ms, and 30 ms, respectively.

FIG. 4 shows representative plots of average oxidation events per lysozyme molecule derived from deconvolved mass spectra for 20 μM lysozyme at 864 μm (●) and 508 μm (♦) aluminum attenuation and 20 μM lysozyme+80 μM GDP at 864 (◯) and 508 (⋄) μm aluminum attenuation.

FIG. 5 shows representative dose-response curves for tryptic peptides obtained for lysozyme at 864 μm (●) and 508 μM (▪) aluminum attenuations. The solid lines represent fits of a single exponential function to each set of points.

FIG. 6 shows a representative deconvolved mass spectrum for 20 μM lysozyme exposed for 20 ms in the presence of 20 mM sodium triflinate at 305 μm aluminum attenuation. Annotation marks depict unmodified lysozyme (#), lysozyme modified by OH only (+), lysozyme modified by CF₃ only ($), and lysozyme modified by a mixture of CF₃ and OH (*).

### DETAILED DESCRIPTION

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices and methods relating to modifying biological molecules are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

Synchrotron X-ray footprinting is a structural biology method used to examine the structure of biomolecules in native solution states. The technique relies upon X-ray radiolysis of water by synchrotron radiation to generate reactive hydroxyl radicals, which then readily react with, for example, the side chains of amino acids in proteins or the phosphodiester backbone of nucleic acids, resulting in their cleavage. In the case of proteins, this reaction occurs at rates proportional to both solvent accessibility of a particular amino acid in the protein, as well as its intrinsic reactivity with hydroxyl radicals. These data are thus a map of solvent water accessibility across the protein, which is then read out by mass spectrometry (MS) methods to identify specific amino acids that were covalently labeled by hydroxyl radicals. Changes in protein structure and thus solvent accessibility in response to an external stimulus such as binding of a ligand, protein folding, or dynamics changes during a reactive transformation can be followed using this method, complementing static data obtained by other methods such as macromolecular crystallography or cryo-electron microscopy. The technique is quite complementary to MS-based hydrogen deuterium exchange, except the side chains are labeled instead of the backbone, and the non-exchangeability of the hydroxyl labels provides a pure solvent accessibility measure.

There are a range of methods to generate hydroxyl radicals for hydroxyl radical mediated protein footprinting (HRPF) besides synchrotron radiation, including benchtop Fenton chemistry, plasma generation, and laser photolysis in the presence of peroxide. These approaches differ in the timescales for production of hydroxyl radicals (from microseconds to seconds) as well as the total achievable radical dose, but all yield reliable and reproducible data despite these variations. Close attention to hydroxyl radical dose and resultant protein effects is key to success in using any HRPF approach, and indeed dosimetry using a range of indirect dosimeters is now a well-established feature of optimization in the method. These dosimetry approaches share a common technical challenge of determining a suitable hydroxyl radical dose ranges on the sample that would yield sufficient labeling coverage to probe regions of interest without damaging or otherwise perturbing the sample state. Such dose range is partially a function of the competition between solvent accessible, reactive hydroxyl radical sites on the target macromolecule and other scavengers in solution which can interfere with the labeling chemistry.

Synchrotron beamlines deliver a measurable, reproducible, and controlled dose of hydroxyl radicals to biological samples in aqueous solution on microsecond to millisecond and longer timescales, with dose based on the sample's X-ray cross section. Their ability to deliver a very high dose of radicals makes such beamlines well suited for studies of complex and highly scavenging systems such as virus assembly or complex mixtures of proteins in neurodegenerative diseases, as well as time-resolved studies of macromolecule dynamics in a complex evolving system, e.g., the early steps in G protein activation by GPCRs or assessing metal ion movement through transport proteins on millisecond timescales. Currently, two dedicated resources for synchrotron X-ray footprinting exist worldwide, both in the United States: the 17-BM beamline at the National Synchrotron Light Source II (NSLS-II) (New York) and beamline 3.3.1 at the Advanced Light Source (California). A new 96-well high-throughput exposure apparatus device for the XFP beamline was previously described, along with a workflow to optimize X-ray dose conditions using an Alexa488 fluorescence assay and a plate reader. This system allows rapid screening of protein targets and candidate buffer conditions in a matter of hours to efficiently optimize sample chemistries for ideal hydroxyl radical labeling. However, the Alexa488 assay is only an indirect measure of putative labeling, and mass spectrometry is necessary to properly quantify the success of the experiment at the beamline. Traditional bottom-up analysis methods with proteolysis and LC-MS are time-consuming and are often carried out at a temporal and spatial remove from the beamline. This can result in non-optimal experimental outcomes, and slows the feedback loop between sample chemistry optimization, beamline experiment, and data analysis. Indeed, in some cases it was observed that presumably ideal Alexa488 hydroxyl radical dose response rates nonetheless resulted in poor labeling efficiencies for a target sample, necessitating reinvestigation of sample preparation and buffer composition weeks or months after the initial experiment.

The present disclosure relates to methods for direct assessment and control of the extent of protein labeling for protein footprinting using covalent labeling reagents including hydroxyl radical. In various embodiments, the present disclosure provides a top-down or intact mass spectrometry-based approach to determine and control the dose of label relative to the protein, e.g., by determining an absolute average number of labels per protein molecule (also referred to as oxidation events or labels per protein). Thus, the method described here may provide a direct and absolute dosimetry for improving all protein footprinting experiments for all types of labeling chemistry including multiplex-labeling (e.g., multiple labels of the same or different mass). In addition, the method described herein may be used as a quantitative metric to assess the amount of labeling observed, hence providing a basis for placing other indirect measures of hydroxyl radical dose reported to date on firmer technical grounds.

Thus, the present disclosure addresses the current challenges of dosimetry evaluation and control in protein footprinting technologies by integrating intact MS screening of labeled samples immediately following label exposure, along with metrics to quantify the extent of observed labeling from the intact mass spectra. As an example, intact MS results on the model protein lysozyme were evaluated in the context of Alexa488 assay results and a bottom-up MS analysis of the same samples. Advantageously, the present method may provide an explicit parameter with which to ensure increasingly successful hydroxyl radical mediated protein footprinting (HRPF) experimental outcomes for a wide range of structural biology problems where HRPF can be usefully applied.

In one aspect, the present disclosure provides a method of analyzing protein structure, the method comprising:
(a) labeling a protein with a label to generate an intact labeled protein, wherein the label comprises a hydroxyl radical;
(b) obtaining a mass spectrum of the intact labeled protein; and
(c) analyzing the mass spectrum of the intact labeled protein.

A “protein” or “polypeptide” is a linked sequence of amino acids linked by peptide bonds. The protein or polypeptide can be a natural or synthetic product, or a modification or combination thereof. Examples of proteins as disclosed herein include, but are not limited to, binding proteins, receptors, and antibodies. The terms “polypeptide” and “protein” may be used interchangeably herein. “Primary structure” refers to the amino acid sequence of a particular protein. “Secondary structure” refers to locally ordered regions of hydrogen bonding (alpha and beta structures) which with tertiary contacts (separated pieces of sequence made adjacent by folding) comprises the three-dimensional structures within a protein. These structures are commonly known as domains, e.g., enzymatic domains, extracellular domains, transmembrane domains, pore domains, and cytoplasmic tail domains. Domains are portions of a protein that form a compact unit of the protein and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a protein sequence, which may be 2 to 20, 2 to 15, or 2 to 10 amino acids in length.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

In some embodiments, the protein of the present disclosure in protein is at least 50 amino acids in length, including for example at least 60 amino acids, at least 70 amino acids, at least 80 amino acids, at least 90 amino acids, at least 100 amino acids, at least 120 amino acids, at least 150 amino acids, or at least 200 amino acids in length. In some embodiments, the protein is a natural protein, such as a protein derived from a biological source. In some embodiments, the protein is a synthetic protein, such as a recombinant protein produced by a recombinant expression system. In some embodiments, the protein is an enzyme. For example, the protein can be a lysozyme, such as lysozyme from chicken egg white.

The term “intact,” “intact protein,” or “intact labeled protein” as used herein can include a protein or a labeled protein, which is not subject to any in vitro treatment (such as proteolysis) that results in truncating or fragmenting the sequence of the protein or the labeled protein into shorter sequences. An intact protein can include all forms of the same protein in a cell, a tissue, or a living organism. As nonlimiting examples, the intact protein can be a cytosolic protein, a membrane-bound protein, a protein with post translational modification, a subunit of a functional protein complex or enzyme. In some embodiments, the protein is isolated from a living cell and the present labeling process modifies the isolated protein with multiple labels (e.g., through covalent bonding) without truncating or fragmenting the protein, thereby producing an intact labeled protein.

Both native and denatured proteins may be used in the present method. In some embodiments, the protein is denatured prior to mass spectrometry. For example, following the labeling step, the intact labeled protein is denatured before being subject to mass spectrometric analysis. In some embodiments, the protein remains an intact labeled protein after denaturation.

The protein of the present disclosure can be labeled, for example, by chemical labeling agents that provide for example, a hydroxy radical. Suitable labeling techniques for the present methods include all known hydroxyl radical labeling reagents and methodologies used for protein footprinting studies as well as labeling by other covalent chemistries. The protein may be labeled by a single type of label or multiple types of labels. As a result, the intact labeled protein as disclosed herein may include one type of label or multiple types of labels. In some embodiments, the label is a combination of a hydroxyl radical and at least one different radical (such as trifluoromethyl radical). In some embodiments, the label consists essentially of a hydroxyl radical. In some embodiments, the label is a hydroxyl radical.

In some embodiments, the label is generated by X-ray radiolysis, such as those employed in synchrotron X-ray footprinting studies. For example, X-ray radiolysis of water may be used to generate reactive hydroxyl radicals that react with solvent accessible side chains of the protein. Suitable methods for generating hydroxyl and other radicals as protein labeling agents include those disclosed in Jain et al. (Journal of Synchrotron Radiation, 2021, 28, 1321-1332) and WO 2022/179024, the contents of which are incorporated by reference herein in their entireties. In some embodiments, the label is a combination of a hydroxyl radical and a trifluoromethyl radical, which can be generated from a trifluoromethyl radical precursor. Suitable trifluoromethyl radical precursors include, but are not limited to, Langlois reagent (sodium trifluoromethanesulfinate, or sodium triflinate), Ruppert's reagent, Togui's Reagent, Umemoto's reagent, triflinate chloride, and other precursors. In some embodiments, the trifluoromethyl radical precursor is Langlois reagent (sodium triflinate), umemato-tetrafluoroborate, umemato-trifluoromethanesulfonate, zinc trifluoromethanesulfonate, ethyl trifluoromethanesulfonate, 4,4,4,4′,4′,4′-hexafluoro-DL-valine, or a combination thereof. In some embodiments, trifluoromethyl radicals are generated from Langlois reagent (sodium triflinate).

The intact labeled protein may be analyzed by mass spectrometry. Suitable mass spectrometry includes all known mass spectrometers and methodologies used for protein analysis. The mass spectrometer can be a high-resolution mass spectrometer. Examples of suitable commercial mass spectrometers include, but are not limited to ThermoFisher Scientific instruments such as Orbitrap Eclipse, Orbitrap Fusion Lumos, Q Exactive Plus, Q Exactive HF and Orbitrap Elite, and Bruker timsTOF Pro mass spectrometer, and Waters SynaptG2. The mass spectra of the intact labeled protein as disclosed herein can be obtained and analyzed using known analytical tool, including various computer software. In some embodiments, the mass spectra of the intact labeled protein are obtained using an ESI-MS mass spectrometer.

In some embodiments, analyzing the mass spectrum of the intact labeled protein includes determining an average number of labels per protein molecule. This can be achieved, for example, by determining a molar ratio between the label moiety identified in the intact labeled protein and the protein being labeled. In some embodiment, an “average oxidation events per protein” (OEP) is derived from the average number of labels (e.g., hydroxyl radical or trifluoromethyl radical labels) per protein molecule in the intact labeled protein. The OEP may be used as a dosimetry parameter to quantitatively assess the intact mass spectra and evaluate the extent of observed labeling, which in turn may provide guidance for further labeling process. The OEP value may depend the structure of the protein being labeled as well as the labeling conditions. The OEP value can be, for example, about 0.1, abut 0.5, about 1, about 2, about 3, about 4, about 5, about 6, about 7, about 8, about 9, about 10, about 20, about 50 or about 100.

In some embodiments, the present method further comprises (d) determining a dose of the label based on the average number of labels per protein molecule. The dose may be in a numeric form representing the molar ratio between the label introduced to the protein in a labeling process and the protein being labeled. For example, an experimental dose of the label for a given protein under certain conditions may be derived from the average number of labels per protein molecule. The experimental dose determined in this process may indicate that the protein is under-labeled (e.g., there remain available reactive sites on the protein to react with more labels) or that the protein has reached a saturated level of labeling (e.g., no increase in the average number of labels per protein molecule is observed despite the use of increased amounts of the label). The experimental dose can then be adjusted to achieve a suitable dose for further labeling process (e.g., to produce the intact labeled protein on a larger scale under the same conditions). In particular, the dose of the label of the present method may be selected to both achieve adequate labeling of the protein (e.g., to ensure detection of labeled products) and avoid excessive exposure to the label that causes damage of the protein. Thus, the experimental dose acquired from the mass spectra of the intact labeled protein may provide guidance for selecting a suitable dose range for improved labeling of the protein in broader applications. This can also be imagined as a method for optimizing synthesis of specific labeled proteoforms.

In some embodiments, the method further comprises (e) repeating steps (a)-(d), in which the protein is labeled with the label according to the dose of the label. For example, following an initial labeling and determination of an experimental dose, the labeling process and mass spectrometric analysis may be repeated to identify a suitable dose range for the protein under set conditions.

In another aspect, the present disclosure provides a method of determining a dose of hydroxyl radicals for protein labeling, the method comprising:
(i) labeling a protein with hydroxyl radicals to generate an intact labeled protein;
(ii) obtaining a mass spectrum of the intact labeled protein; and
(iii) analyzing the mass spectrum of the intact labeled protein to determine the dose of hydroxyl radicals.

In some embodiments, the method of determining a dose of hydroxyl radicals for protein labeling further comprises (iv) repeating (i)-(iii) to obtain a range of doses of hydroxyl radicals. The range of doses may include a dose by which the protein is under-labeled and/or dose by which the protein has reached a saturated level of labeling. As described herein, determination the dose or dose range may include measurement of an average number of labels per protein molecule, which can be obtained by analysis of a mass spectrum of the labeled protein. In some embodiments, analyzing the mass spectrum of the intact labeled protein to determine the dose of hydroxyl radicals comprises determining an average number of hydroxyl radicals per protein molecule.

In another aspect, the present disclosure provides a method of labeling a protein, the method comprising:
labeling a protein with a label to generate an intact labeled protein, wherein the label comprises a hydroxyl radical;
subjecting the intact labeled protein to mass spectrometry, thereby determining a dose of the label relative to the protein; and
further labeling the protein with the label according to the dose of the label.

The protein labeling method as described herein can produce intact labeled proteins with desirable qualities, including improved detection capacity due to sufficient level of labels in the labeled protein and intact structure without damage caused by excessive amount of reactive labels.

In another aspect, the present disclosure provides an intact labeled protein produced by the protein labeling method as described herein. For example, the intact labeled protein produced by the present method can be an intact labeled lysozyme, such as lysozyme from chicken egg white.

In another aspect, the present disclosure provides a method of performing protein footprinting, the method comprising:
labeling a protein according to the protein labeling method as described herein to generate a labeled protein sample; and
analyzing the labeled protein sample, thereby a structural character of the protein is determined.

In some embodiments, the labeled protein sample for performing protein footprinting is analyzed using mass spectrometry. For the protein footprinting processes, both top-down and both-up methodologies can be used. For example, once the intact labeled protein is obtained as disclosed herein, the labeled protein can be analyzed in its intact form (top-town measurement) or be subject to proteolysis and subsequent bottom-up analysis of the resulting protein fragments. In some embodiments, analyzing the labeled protein sample for the present protein footprinting method comprises: (i) analyzing the intact labeled protein in the labeled protein sample using mass spectrometry, (ii) digesting the intact labeled protein in the labeled protein sample into fragments and analyzing the fragments using mass spectrometry, or both. In some embodiments, the method comprises digesting the intact labeled protein in the labeled protein sample into fragments and analyzing the fragments using mass spectrometry. The analytical results of the protein fragments may be used, for example, to validate the results (such as OEP) obtained from analysis of the intact labeled protein. Together, the top-down and both-up approaches based on the intact labeled protein prepared by the present disclosure may provide corroborating and/or complementary structural information of the intact protein.

The present protein footprinting method may be used to uncover or monitor structural features of the protein of interest, including but not limited to, ligand binding, active site, protein folding, and solvent accessibility.

### Example 1

A mass spectrometry instrument was installed near the X-ray Footprinting of Biological Materials (XFP) beamline in NSLS-II laboratory. Access to mass spectrometry near the beamline offers an opportunity to develop approaches to evaluate hydroxyl radical labeling efficiency shortly after X-ray exposure while an investigator is still present at the NSLS-II and able to refine their conditions based on the outcome of the mass spectrometry screening. In addition, this resource permits new dose metrics based directly on mass spectrometric data that can be used for experiment design.

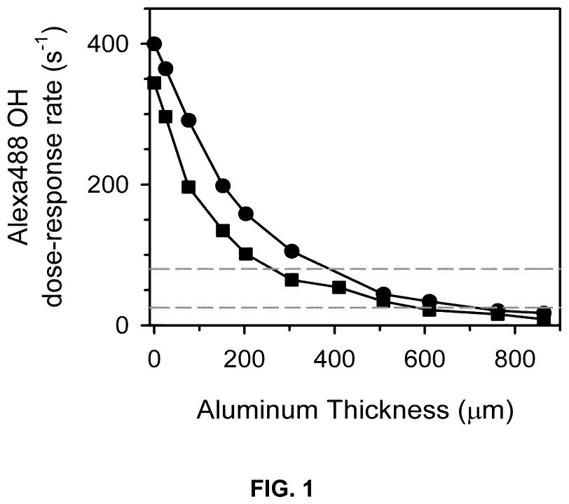

In this example, intact mass spectrometry approaches were employed to rapidly assess the outcome of X-ray footprinting experiments at the XFP beamline using lysozyme as a simple test protein. These top-down results are compared to Alexa488 dose-response data and bottom-up proteomics of the same samples. Based on these studies, a new direct dosimetry parameter termed “average oxidation events per protein” may be used to quantitatively assess the intact mass spectra and evaluate the extent of observed labeling prior to more detailed mass spectrometric measurements and data analysis. This work may provide a framework for a standardized workflow that can be applied to a significant fraction of protein X-ray footprinting experiments at the NSLS-II XFP beamline.
Materials. Lysozyme from chicken egg white (≥98% purity) and guanosine diphosphate (GDP) were purchased from MilliporeSigma (Burlington, MA). LC-MS and HPLC grade water and acetonitrile were purchased from Honeywell International Inc. (Charlotte, NC).
Synchrotron X-ray Footprinting Experiments. X-ray exposure of samples was carried out at the X-ray Footprinting of Biological Materials (XFP) beamline located at the National Synchrotron Light Source II (NSLS-II, Brookhaven National Laboratory, Upton, NY), using a 96-well high-throughput apparatus at a constant temperature of 25° C. and ring current of 400 mA. Incident beam powers on the samples as a function of attenuation were determined as described previously. Solution samples were exposed in 5 μL sample droplets held by surface tension on the bottom of 200 μL PCR tubes. A previously reported Alexa488-based assay that uses the loss of fluorescence following X-ray exposure was used to evaluate hydroxyl radical dose for a range of exposure times and attenuation conditions in order to select conditions for exposure. Alexa488 was added to a concentration of 4 μM, and fluorescence was measured before and after exposure directly in the PCR tubes using a Biotek Synergy HIM plate reader. The loss of fluorescence as a function of exposure time was fit to a first order rate equation to extract an Alexa488 hydroxyl radical dose response rate. Subsequent X-ray exposures of 20 μM lysozyme samples (in 10 mM sodium phosphate pH 7.4 buffer) in the presence and absence of 80 μM GDP for mass spectrometry analysis were carried out at two different X-ray flux conditions using 864 μm and 508 μm thick aluminum attenuators, for 0, 12, 20, and 30 ms exposure times. Following exposures, samples were immediately quenched using methionine amide to a final concentration of 10 mM, flash frozen in liquid nitrogen, and stored at −80° C.
Intact Mass Spectrometry Data Collection & Data Analysis. Exposed lysozyme samples were desalted and exchanged into a denaturing solvent (60% acetonitrile/40% water/0.1% formic acid) using MonoSpin Reversed Phase C18 spin columns (GL Sciences Inc, CA). ESI-MS spectra were acquired in positive mode by direct infusion at a flow rate of 5 μL/min using a HESI-II source on an Orbitrap Elite mass spectrometer (Thermo Scientific, CA). Mass spectra were acquired for a 60 second accumulation for each sample using a m/z range of 500-2000, with FT resolution set to 120000, a source voltage of 3.5 kV, a source temperature of 50° C. Deconvolution of the intact mass spectra was carried out using Unidec.
Bottom-up Mass Spectrometry and Data Analysis. Exposed lysozyme samples (2.8 μg in 10 μL) were dried completely via speed-vacuum and reconstituted in 20 mM Tris/6 μM urea buffer (pH 8.0). These samples were then reduced with 10 mM dithiothreitol (DTT) at 37° C. for 45 min and alkylated with 25 mM iodoacetamide at room temperature for 1 hour in the dark. Protein samples were then digested with trypsin (Promega, MA) at 37° C. overnight using a 1:10 enzyme:protein molar ratio. The digestion reaction was terminated by adding 5% formic acid to a final concentration of 0.1%. Identification and quantification of oxidative sites were performed by nano-LC-MS analysis using an Orbitrap Eclipse mass spectrometer (Thermo Scientific, CA) interfaced with a Waters nanoAcquity UPLC system (Waters, MA). Digested peptides (˜600 ng in 8 μl; ˜300 ng in 4 μl) were loaded on a trap column (180 μm×20 mm packed with C18 Symmetry, 5 μm, 100 Å; Waters, MA) to desalt and concentrate peptides. The peptide mixture was separated on a reverse phase column (75 μm×250 mm column packed with C18 BEH, 1.7 μm, 300 Å; Waters, MA) using a linear gradient of 0 to 32% mobile phase B (100% acetonitrile/0.1% formic acid) vs. mobile phase A (100% water/0.1% formic acid) for 60 minutes at 40° C. at a flow rate of 300 nL/min. Eluted peptides were introduced into the nano-electrospray source at a capillary voltage of 2.0 kV. MSI spectra were acquired in the Orbitrap Eclipse (R=120K: AGC target=400,000; MaxIT=auto; RF Lens=30%; mass range=360-1600) for eluted peptides. MS/MS spectra were collected in the linear ion trap (rate turbo); AGC target 10,000; MaxIT=35 ms; NCECID=35%). The resulting MS/MS spectra were searched for peptides generated by trypsin digestion from the lysozyme protein sequence using MassMatrix to identify specific sites of modification. Mass accuracy values of 10 ppm and 0.8 Daltons were used for MSI and MS/MS scans respectively, with allowed variable modifications for all known oxidative modifications previously documented for amino acid side chains. MS/MS spectra for each site of proposed modification were manually validated. The abundance of unmodified and oxidized species for each peptide was determined by extracting and integrating their selected ion chromatograms using XCalibur. The fraction of unmodified peptide for each exposure time was calculated based on previous approaches. Dose-response curves were generated by plotting the fraction of unmodified peptide versus exposure time and fitting the data to a single exponential equation using Origin.
Results. This study explored intact mass spectrometry screening of lysozyme under different X-ray dose conditions. In synchrotron X-ray footprinting experiments, the exposure time and X-ray flux incident on the sample are variables for controlling the total X-ray dose, and thus yield of hydroxyl radicals available to the sample. A key determinant for experiment outcome is selection of an X-ray dose to maximize labeling of the solvent-accessible amino acids while also avoiding excessive non-specific radiation damage that destroys the protein target of interest. In order to identify a range of conditions for developing analysis in these studies, the impact of X-ray flux was evaluated by varying attenuation and scavenging, using a previously reported Alexa488-based hydroxyl radical dose-response assay for both 20 μM lysozyme alone and 20 μM lysozyme in the presence of 80 μM GDP acting as a hydroxyl radical scavenging compound. The addition of a scavenger such as GDP suppresses the Alexa hydroxyl radical dose-response rate constant relative to that observed for lysozyme alone (FIG. 1), consistent with GDP acting as a sponge for available hydroxyl radicals in solution. Empirical observations over the past several years at the XFP beamline show that, in the context of actual experimental conditions of protein and buffer concentrations, adjusting the attenuation to achieve an Alexa hydroxyl radical dose response rate constants of 25-80 s⁻¹ will generally provide good labeling coverage without excessive radiation damage for exposure times ranging from 0-50 ms for a wide range of protein systems less than 150 kDa. For this study, two different attenuation conditions were selected for the 14.7 kDa lysozyme protein to evaluate the validity of this assumption. The first, using 864 μm aluminum, provides a low dose condition having Alexa hydroxyl radical dose-response rate constants somewhat below the empirical threshold for good labeling, with rates of 17.4 s⁻¹ for lysozyme alone and 8.5 s⁻¹ for lysozyme and GDP. The second condition using 508 μm aluminum gives Alexa488 hydroxyl radical dose-response rates within the desired range (44.3 s⁻¹ and 34.0 s⁻¹, respectively). Both 20 μM lysozyme alone and 20 μM lysozyme in the presence of 80 μM GDP were exposed to X-rays at each attenuation condition for 0, 12, 20, and 30 milliseconds, buffer exchanged into denaturing solvent, and then evaluated via ESI-MS using direct infusion methods.

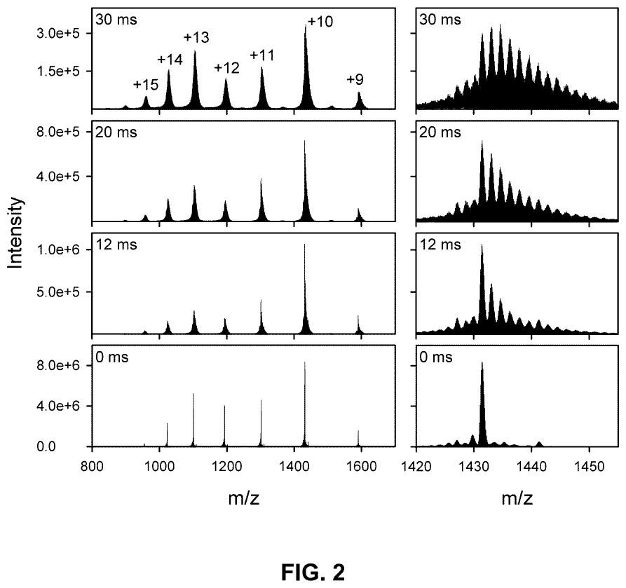

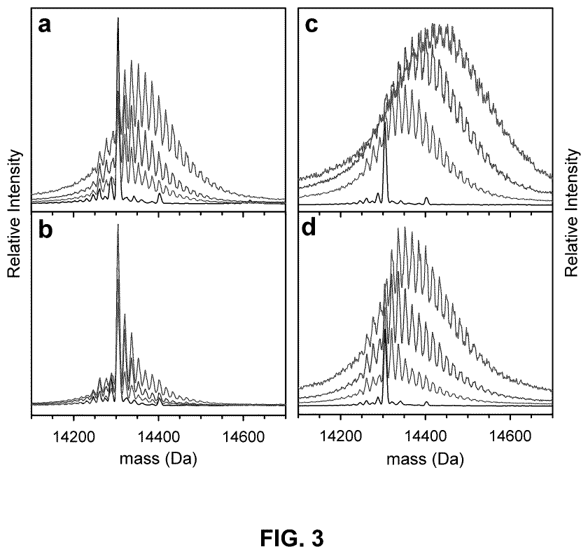

FIG. 2 depicts representative intact mass spectra obtained under denaturing conditions for lysozyme at 864 μm aluminum attenuation for several different X-ray exposure times. Seven distinct charge states are clearly visible, with the +10 charge state being most intense in all cases. Notably, spectra show a significant decrease in TIC intensity with increasing exposure time. Deconvolution of the unexposed 0 ms sample yields a spectrum (FIG. 3A) centered around an intense peak at mass 14309.3 Da, supporting its assignment to unmodified chicken egg white lysozyme (molecular weight 14307 Da). With increasing X-ray exposure, a series of new peaks at higher m/z appear that are separated from one another by 16 Da (FIG. 3A). Concurrently, the intensity of the parent 14309.3 Da unmodified lysozyme signal decreases with increased X-ray dose. This observation is consistent with progressive addition of oxygen to lysozyme via X-ray induced hydroxyl-radical labeling with increasing X-ray exposure, and is in accord with spectra obtained during prior proof of concept studies of HRPF using X-rays or laser photolysis. The deconvolved mass spectra for lysozyme in the presence of GDP as a scavenger show significantly less intense+16 satellite peaks relative to the unmodified lysozyme molecule (FIG. 3B), consistent with a lowered extent of oxidation and in good agreement with the smaller Alexa488 hydroxyl radical dose-response rate seen for this sample compared to lysozyme alone. In contrast, using 508 μm aluminum attenuation to provide higher X-ray flux yields substantially more oxidation in both samples (FIGS. 3C and 3D) relative to the low-flux condition. Indeed, the deconvoluted spectra for lysozyme alone at 508 μm aluminum show rather poor signal to noise and decreased peak-to-peak resolution as total exposure time is increased. This may indicate increasingly complex X-ray induced modifications as well as excessive non-specific protein damage.

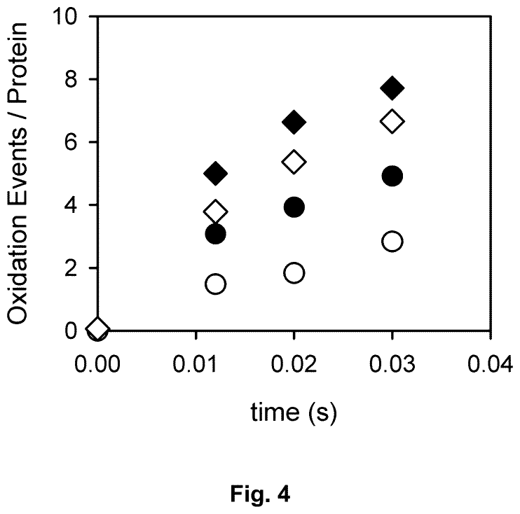

An average “oxidation events per protein” (OEP) was used as a parameter to quantitatively evaluate the extent of oxidation at each condition in the intact mass spectra, which is defined by the weighted average of the peak heights for the series of peaks seen in the deconvoluted spectra stemming from +16 Da addition of oxygen to protein, assuming equal ionization efficiencies for the unmodified and modified forms. Plots of OEP values as a function of time (FIG. 4) show that the observed extent of lysozyme oxidation matches the trend seen in Alexa488 hydroxyl dose-response rates, supporting the notion that the Alexa488 assay is an accurate although indirect predictor for labeling extent. Average OEP numbers range from 1 to 2 for lysozyme in the presence of GDP under a low X-ray dose condition with 864 μm aluminum up to values of 5 to 8 for lysozyme alone with 508 μm aluminum (a high dose condition). Data points in the 12-30 ms window appear roughly linear following a more rapid rise from 0 to 12 ms, and the rate of increase in OEP is greater for 508 μm aluminum attenuation, consistent with a more rapid accumulation of dose over time compared to that seen with 864 μm aluminum attenuation. In principle, the OEP numbers should rise to a theoretical upper limit at very high X-ray dose that is constrained by the solvent accessible surface residues of a particular protein as well as their relative intrinsic reactivities towards hydroxyl radicals. However, it is estimated that under these conditions determining this experimentally may be challenging due to increasing levels of nonspecific protein damage and increasingly poor signal-to-noise resolution at high dose, as suggested by the lysozyme spectra obtained with 508 μm aluminum attenuation.

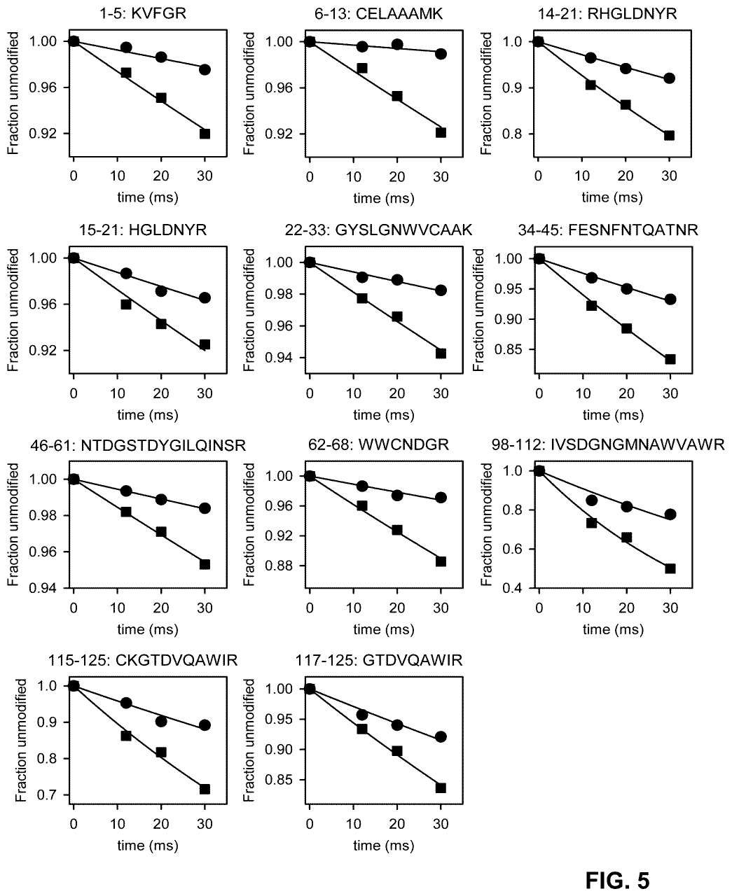

The intact mass spectrometry screening approach was validated by conventional bottom-up assessment of the exposed lysozyme samples obtained at both low and high hydroxyl radical doses. Evaluation of the nano-LC-MS data obtained following trypsin digestion of exposed lysozyme at both conditions revealed 11 distinct tryptic peptides covering ˜74% of the lysozyme sequence, each of which showed detectable oxidation of one or more amino acid residues. Dose-response rates for each peptide were determined by fitting the fraction of unmodified peptide found via mass spectrometry at each exposure time (0-30 ms) to a first-order rate equation. This provides a measure of the reactivity of a given region of the protein relative to other regions of the protein as well as in response to changes in either structure or total X-ray dose. The observed dose response rates for peptide modifications increase by a factor of 2.75±0.51 for the high-dose condition (508 μm aluminum) compared to the low-dose condition using 864 μm aluminum (Table 1, note that this excludes one peptide showing an 8.8-fold increase, which was attributed to methionine over-oxidation). For both conditions, the dose response curves show no deviations from pseudo-first-order kinetics, indicating no significant effects from over-oxidation or reaction with secondary radicals (FIG. 5). The observed increase in dose-response rates is in excellent agreement with the ratio of 2.55 seen for Alexa488 hydroxyl dose-response rates (44.3 s⁻¹ for 508 μm aluminum and 17.4 s⁻¹ for 864 μm aluminum) as well as the ratio of 2.40 anticipated for calculated incident beam power available to the sample (6.0 W for 508 μm aluminum and 2.5 W for 864 μm aluminum, at NSLS-II design currents of 500 mA), further supporting the notion that the Alexa488 dose response assay is an accurate predictor of hydroxyl labeling under these conditions. Identical labeling coverage was obtained at both dose conditions, apart from a single additional labeled residue at the 508 μm aluminum condition (Table 1). This implies that the increased OEP values enabled by increased X-ray dose under these conditions for lysozyme manifests almost entirely as a larger fractional modification of the same set of solvent accessible amino acids, as opposed to labeling of new amino acids that did not show detectable levels of labeling under a lower X-ray dose. It is possible that even higher X-ray doses would lead to enhanced hydroxyl radical labeling coverage, but this would come at the expense of possible overoxidation and protein damage from secondary radical reactions. Alternative strategies, such as multiplex labeling with trifluoromethyl radical chemistry, would be a more effective approach to enhance labeling coverage while ensuring the X-ray dose is properly poised to limit excess radiation damage.

TABLE 1
Observed lysozyme peptide oxidative modification
rates at two X-ray dose conditions.

| | | 864 μm Al | 508 μm Al | |
|---|---|---|---|---|
| peptide | sequence ^{a} | rate (s⁻¹) | rate (s⁻¹) | ratio |
| 1-5 | KVFGR | 0.752 ± 0.070 | 2.66 ± 0.10 | 3.54 |
| 6-13 | CELAAAMK | 0.291 ± 0.062 | 2.56 ± 0.15 | 8.80 |
| 14-21 | RHGLDNYR | 2.84 ± 0.07 | 7.59 ± 0.15 | 2.67 |
| 15-21 | HGLDNYR | 1.24 ± 0.08 | 2.78 ± 0.16 | 2.24 |
| 22-33 | GYSLGNWVCAAK | 0.601 ± 0.038 | 1.90 ± 0.06 | 3.16 |
| 34-45 | FESNFNTQATNR | 2.42 ± 0.08 | 6.16 ± 0.13 | 2.55 |
| 46-61 | NTDGSTDYGILQINSR | 0.546 ± 0.006 | 1.56 ± 0.04 | 2.86 |
| 62-68 | WWCNDGR | 1.09 ± 0.09 | 3.89 ± 0.13 | 3.57 |
| 98-112 | IVSDGNGMNAWVAWR | 9.57 ± 0.99 | 22.78 ± 1.02 | 2.38 |
| 115-125 | CKGTDVQAWIR | 4.22 ± 0.34 | 10.97 ± 0.42 | 2.60 |
| 117-125 | GTDVQAWIR | 2.94 ± 0.17 | 5.76 ± 0.14 | 1.96 |

^{a} Sites of modification observed at both dose conditions marked in bold text; sites seen only at the 508 μm aluminum condition marked in italics.

Discussion Successful synchrotron HRPF experiments rely on selecting an appropriate X-ray dose to adequately label the sample in solvent accessible regions of interest. A rapid screening assay developed some years ago that relies on loss of Alexa488 fluorescence due to hydroxyl radicals has proven to be an excellent proxy for dose but suffers from being an indirect measure. Here, a process to assess the extent of covalent labeling using standard intact mass spectrometry methods was carried out under the conditions described in this Example. Sample exchange into a denaturing solvent and mass spectrometry data acquisition can be completed within a matter of hours following sample exposure at the beamline, providing rapid feedback to experimenters on whether there was optimal labeling of the sample and whether further refinement is needed prior to embarking on exposure of samples for a detailed bottom-up LC-MS/MS characterization. In principle, measuring mass spectra under native conditions via direct infusion could eliminate the buffer exchange step to save more time. However, this requires using a suitable volatile buffer such as ammonium acetate during sample exposure. Most HRPF experiments employ non-scavenging and non-volatile PBS buffer at physiological pH with significant concentrations of salt and organic buffer additives that are not suitable for direct infusion into a mass spectrometer, and thus the buffer exchange step is still required. Moreover, native conditions yield mass spectra having high m/z values and low charge, which requires mass analyzers capable of very high m/z to successfully observe even relatively modest sized proteins (the Orbitrap Elite instrument available at NSLS-II is limited to m/z<4000). The use of denaturing conditions surmounts this issue due to increased average ion charge, albeit with a greater distribution of charge states than is the case for native conditions.

A new hydroxyl radical dose parameter, average oxidation events per protein (OEP), can be used to evaluate intact mass spectra through this method. With this approach, it was shown that dose conditions well below empirical Alexa488 hydroxyl radical dose response rate threshold of 25 s⁻¹ can yield sufficient modification of lysozyme for detailed analysis, which is corroborated by bottom-up analysis. This highlights the value in assessing the extent of labeling by direct mass spectrometry methods as a routine part of the experiment. The strong correlation between extent of labeling and the indirect Alexa hydroxyl radical dose response rates seen in this study may be used to devise more quantitative rules for optimal Alexa488 dose response rates, by evaluating OEP parameters for a range of dose-response rates for proteins spanning a wide range of molecular weights and compositions. It also demonstrates that rather modest X-ray doses leading to addition of ca. 2 oxygens per protein (or approximately 2 oxygens/100 residues as chicken egg white lysozyme has 129 residues) can provide excellent labeling coverage for X-ray footprinting of soluble proteins, reducing the risk of working at a dose where secondary radical reactions convolute the analysis. Conversely, conditions yielding >5 oxygens per 100 residues also give reliable outcomes, suggesting that useful synchrotron HRPF biophysical data can be obtained over a fairly large span of hydroxyl radical doses, in line with observations made decades ago for DNase footprinting of DNA-protein interactions. This can be rationalized by the location and chemical nature of the modifications. The modified residues are typically on solvent accessible surfaces of the protein, and the modifications are functionally conservative in this environment, e.g., covalent labeling of a Phe residue is equivalent to a Phe to Tyr mutation, slightly increasing the polarity of residues that are, by definition, solvent accessible.

It is noted that OEP values are not an absolute predictive parameter, as it will depend on protein size, the fraction of the protein that is solvent exposed, and the relative reactivities of those solvent-exposed residues towards hydroxyl radicals. Recent advances in structure prediction coupled to experimentally known amino acid sidechain reactivities raise the prospect of new computational tools to feasibly predict achievable OEP numbers as part of the design of an X-ray footprinting study. These parameters, coupled to a more quantitative assessment of the required Alexa488 dose response based on protein size, may yield boundary conditions for the required experimental dose, further speeding up the beamline component of the X-ray footprinting experiment while enabling increased assurances of success.

To conclude, a strategy was developed here to routinely integrate mass spectrometry screening with a synchrotron X-ray footprinting beamline using a reliable top-down measurement. This provides a workflow to rapidly screen experimental outcomes and quantitatively assess the extent of radiolytic oxidation. This methodology may be extended to a wider range of proteins and different labeling chemistries (such as trifluoromethyl radical) and may be carried out with increased throughput of the mass spectrometry step using liquid chromatography or other automation methods.

### Example 2

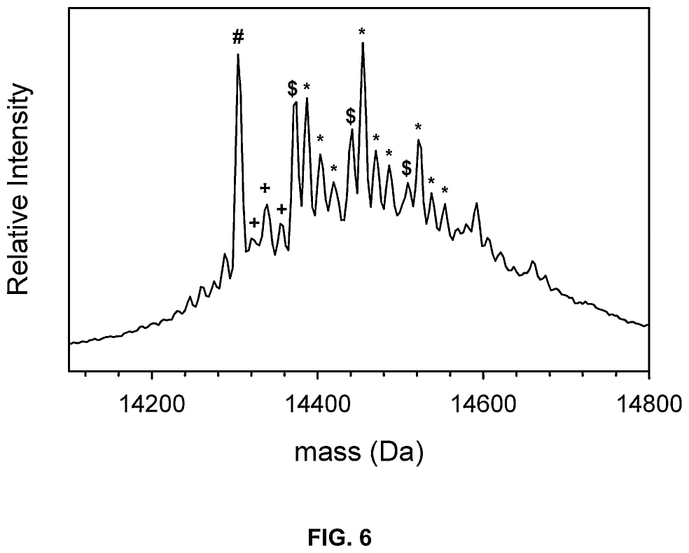

Multiplex labeling. The present method can also be applied to other footprinting labeling chemistries, such as trifluoromethylation, in which exposure of sodium triflinate in solution to laser photolysis or X-rays generates a CF₃ radical which then covalently labels protein side chains. FIG. 6. depicts the results of a “one-pot” labeling experiment in which lysozyme was exposed to X-rays in the presence of 20 mM sodium triflinate. The deconvolved mass spectrum shows the presence of intact unlabeled lysozyme (marked with #) as well as labeling by OH alone (addition of 16 Da to intact lysozyme, marked with +). An additional set of peaks separated from one another by 68 Da can also be seen, corresponding to the addition of a CF₃ moiety to lysozyme. In addition, the +16 Da addition of hydroxyl radical to a CF₃-modified lysozyme molecule can also be detected (peaks marked with *). Analysis of this spectrum using our OEP metric indicates an OEP value of 5.7 OH/lysozyme as well as approximately 0.5 CF₃/lysozyme. This illustrates how the intact mass spectrometry dosimetry method described here can be used to rapidly assess the extent of labeling of proteins using multiple covalent labeling reagents.

### Example 3

Cellular Footprinting. The intact dosimetry method can also be applied to footprinting of intact, live cells under in vivo conditions, in order to optimize the dose needed to obtain significant labeling of a large fraction of proteins within the cell. E. coli is grown in liquid media, aliquots are extracted during log phase growth, and are immediately subjected to X-ray exposure using the NSLS-II 17-BM beamline for 50 ms exposure using 25, 76, and 152 μm aluminum attenuations. Following exposure, cells are lysed to extract the soluble proteins in the cytoplasm, and the soluble material is diluted 100-fold and exchanged into a denaturing solvent mixture (50% acetonitrile/50% water/0.1% formic acid is suitable) for direct injection into the mass spectrometer. Mass spectra are measured via direct infusion ESI-MS of the denatured cell extract, and detectable spectra are obtained for the 3-5 most abundant proteins present in the mixture, which vary based on the growth phase and any nutrient deficiencies of the cells. Deconvolution is performed on the mass spectra and OEP parameters are extracted. The results show that the ribosomal protein elongation factor Tu is the most abundant protein during log phase growth, though it decreases in abundance as the cell culture reaches the stationary phase. More importantly, OEP analysis shows <1 oxygen added/protein for the exposure performed at 152 μm aluminum, whereas the 25 μm and 76 μm aluminum exposures showed OEP values >3. This suggests insufficient oxidative labeling within the cell using 152 μm aluminum attenuation, and therefore exposures using 25 μm and 76 μm aluminum attenuations were selected for further bottom-up LC-MS proteomic analysis.

The present disclosure has described one or more preferred embodiments, and it should be appreciated that many equivalents, alternatives, variations, and modifications, aside from those expressly stated, are possible and within the scope of the invention.

### REFERENCES

[1] J. Kiselar, M. R. Chance, High-Resolution Hydroxyl Radical Protein Footprinting: Biophysics Tool for Drug Discovery, Annual Review of Biophysics 47 (2018) 315-333. 10.1146/annurev-biophys-070317-033123.
[2] A. Mckenzie-Coc, N. S. Montes, L. M. Jones, Hydroxyl Radical Protein Footprinting: A Mass Spectrometry-Based Structural Method for Studying the Higher Order Structure of Proteins, Chemical Reviews 122 (2022) 7532-7561. 10.1021/acs.chemrev.1c00432.
[3] M. R. Chance, E. R. Farquhar, S. Yang, D. T. Lodowski, J. Kisclar, Protein Footprinting: Auxiliary Engine to Power the Structural Biology Revolution, Journal of Molecular Biology 432 (2020) 2973-2984. https://doi.org/10.1016/j.jmb.2020.02.011.
[4] X. Zheng, P. L. Wintrode, M. R. Chance, Complementary Structural Mass Spectrometry Techniques Reveal Local Dynamics in Functionally Important Regions of a Metastable Serpin, Structure 16 (2008) 38-51. https://doi.org/10.1016/j.str.2007.10.019.
[5] B. B. Minkoff, J. M. Blatz, F. A. Choudhury, D. Benjamin, J. L. Shohet, M. R. Sussman, Plasma-Generated OH Radical Production for Analyzing Three-Dimensional Structure in Protein Therapeutics, Scientific Reports 7 (2017) 12946. 10.1038/s41598-017-13371-7.
[6] X. R. Liu, M. M. Zhang, M. L. Gross, Mass Spectrometry-Based Protein Footprinting for Higher-Order Structure Analysis: Fundamentals and Applications, Chemical Reviews 120 (2020) 4355-4454. 10.1021/acs.chemrev.9b00815.
[7] R. Jain, D. Abel, M. Rakitin, M. Sullivan, D. T. Lodowski, M. R. Chance, E. R. Farquhar, New high-throughput endstation to accelerate the experimental optimization pipeline for synchrotron X-ray footprinting, Journal of Synchrotron Radiation 28 (2021) 1321-1332. doi: 10.1107/S1600577521005026.
[8] J. S. Sharp, S. K. Misra, J. J. Persoff, R. W. Egan, S. R. Weinberger, Real Time Normalization of Fast Photochemical Oxidation of Proteins Experiments by Inline Adenine Radical Dosimetry, Analytical Chemistry 90 (2018) 12625-12630. 10.1021/acs.analchem.8b02787.
[9] S. Tetter, N. Terasaka, A. Steinauer, R. J. Bingham, S. Clark, A. J. P. Scott, N. Patel, M. Leibundgut, E. Wroblewski, N. Ban, P. G. Stockley, R. Twarock, D. Hilvert, Evolution of a virus-like architecture and packaging mechanism in a repurposed bacterial protein, Science 372 (2021) 1220-1224. doi: 10.1126/science.abg2822.
[10] M. K. Siddiqi, C. Kim, T. Haldiman, M. Kacirova, B. Wang, J. Bohon, M. R. Chance, J. Kiselar, J. G. Safar, Structurally distinct external solvent-exposed domains drive replication of major human prions, PLOS Pathogens 17 (2021) e1009642. 10.1371/journal.ppat.1009642.
[11] Y. Du, N. M. Duc, S. G. F. Rasmussen, D. Hilger, X. Kubiak, L. Wang, J. Bohon, H. R. Kim, M. Wegrecki, A. Asuru, K. M. Jeong, J. Lec, M. R. Chance, D. T. Lodowski, B. K. Kobilka, K. Y. Chung, Assembly of a GPCR-G Protein Complex, Cell 177 (2019) 1232-1242.c1211. https://doi.org/10.1016/j.cell.2019.04.022.
[12] S. Gupta, J. Chai, J. Cheng, R. D'Mello, M. R. Chance, D. Fu, Visualizing the kinetic power stroke that drives proton-coupled zinc (ii) transport, Nature 512 (2014) 101-104. 10.1038/nature13382.
[13] A. Asuru, E. R. Farquhar, M. Sullivan, D. Abel, J. Toomey, M. R. Chance, J. Bohon, The XFP (17-BM) beamline for X-ray footprinting at NSLS-II, Journal of Synchrotron Radiation 26 (2019) 1388-1399. doi: 10.1107/S1600577519003576.
[14] S. Gupta, R. Celestre, C. J. Petzold, M. R. Chance, C. Ralston, Development of a microsecond X-ray protein footprinting facility at the Advanced Light Source, J Synchrotron Radiat 21 (2014) 690-699. 10.1107/S1600577514007000.
[15] S. Gupta, M. Sullivan, J. Toomey, J. Kiselar, M. R. Chance, The Beamline X28C of the Center for Synchrotron Biosciences: a national resource for biomolecular structure and dynamics experiments using synchrotron footprinting, J Synchrotron Radiat 14 (2007) 233-243. 10.1107/S0909049507013118.
[16] M. T. Marty, A. J. Baldwin, E. G. Marklund, G. K. A. Hochberg, J. L. P. Benesch, C. V. Robinson, Bayesian Deconvolution of Mass and Ion Mobility Spectra: From Binary Interactions to Polydisperse Ensembles, Analytical Chemistry 87 (2015) 4370-4376. 10.1021/acs.analchem.5b00140.
[17] H. Xu, M. A. Freitas, MassMatrix: A database search program for rapid characterization of proteins and peptides from tandem mass spectrometry data, PROTEOMICS 9 (2009) 1548-1555. https://doi.org/10.1002/pmic.200700322.
[18] J. G. Kiselar, S. D. Maleknia, M. Sullivan, K. M. Downard, M. R. Chance, Hydroxyl radical probe of protein surfaces using synchrotron X-ray radiolysis and mass spectrometry, International Journal of Radiation Biology 78 (2002) 101-114. 10.1080/09553000110094805.
[19] D. M. Hambly, M. L. Gross, Laser flash photolysis of hydrogen peroxide to oxidize protein solvent-accessible residues on the microsecond timescale, Journal of the American Society for Mass Spectrometry 16 (2005) 2057-2063. 10.1016/j.jasms.2005.09.008.
[20] R. Jain, N. S. Dhillon, E. R. Farquhar, B. Wang, X. Li, J. Kiselar, M. R. Chance, Multiplex Chemical Labeling of Amino Acids for Protein Footprinting Structure Assessment, Analytical Chemistry 94 (2022) 9819-9825. 10.1021/acs.analchem.2c01640.
[21] M. Brenowitz, D. F. Senear, M. A. Shea, G. K. Ackers, [9] Quantitative DNase footprint titration: A method for studying protein-DNA interactions, Methods in Enzymology, Academic Press 1986, pp. 132-181.
[22] J. Jumper, R. Evans, A. Pritzel, T. Green, M. Figurnov, O. Ronneberger, K. Tunyasuvunakool, R. Bates, A. Žídek, A. Potapenko, A. Bridgland, C. Meyer, S. A. A. Kohl, A. J. Ballard, A. Cowie, B. Romera-Paredes, S. Nikolov, R. Jain, J. Adler, T. Back, S. Petersen, D. Reiman, E. Clancy, M. Zielinski, M. Steinegger, M. Pacholska, T. Berghammer, S. Bodenstein, D. Silver, O. Vinyals, A. W. Senior, K. Kavukcuoglu, P. Kohli, D. Hassabis, Highly accurate protein structure prediction with AlphaFold, Nature 596 (2021) 583-589. 10.1038/s41586-021-03819-2.
[23] G. Xu, M. R. Chance, Radiolytic Modification and Reactivity of Amino Acid Residues Serving as Structural Probes for Protein Footprinting, Analytical Chemistry 77 (2005) 4549-4555. 10.1021/ac050299+.

1. A method of analyzing protein structure, the method comprising:
(a) labeling a protein with a label to generate an intact labeled protein, wherein the label comprises a hydroxyl radical;
(b) obtaining a mass spectrum of the intact labeled protein; and
(c) analyzing the mass spectrum of the intact labeled protein.

2. The method of claim 1, wherein analyzing the mass spectrum of the intact labeled protein comprising determining an average number of labels per protein molecule.

3. The method of claim 1, further comprising
(d) determining a dose of the label based on the average number of labels per protein molecule.

4. The method of claim 3, further comprising
(e) repeating steps (a)-(d), in which the protein is labeled with the label according to the dose of the label.

5. A method of determining a dose of hydroxyl radicals for protein labeling, the method comprising:
(i) labeling a protein with hydroxyl radicals to generate an intact labeled protein;
(ii) obtaining a mass spectrum of the intact labeled protein; and
(iii) analyzing the mass spectrum of the intact labeled protein to determine the dose of hydroxyl radicals.

6. The method of claim 5, further comprising:
(iv) repeating (i)-(iii) to obtain a range of doses of hydroxyl radicals.

7. The method of claim 5, wherein (iii) analyzing the mass spectrum of the intact labeled protein to determine the dose of hydroxyl radicals comprises determining an average number of hydroxyl radicals per protein molecule.

8. A method of labeling a protein, the method comprising:
labeling a protein with a label to generate an intact labeled protein, wherein the label comprises a hydroxyl radical;
subjecting the intact labeled protein to mass spectrometry, thereby determining a dose of the label relative to the protein; and
further labeling the protein with the label according to the dose of the label.

9. A method of performing protein footprinting, the method comprising:
labeling a protein according to the method of claim 8 to generate a labeled protein sample; and
analyzing the labeled protein sample, thereby a structural character of the protein is determined.

10. The method of claim 9, wherein the labeled protein sample is analyzed using mass spectrometry.

11. The method of claim 9, wherein analyzing the labeled protein sample comprises: (i) analyzing the intact labeled protein in the labeled protein sample using mass spectrometry, (ii) digesting the intact labeled protein in the labeled protein sample into fragments and analyzing the fragments using mass spectrometry, or both.

12. The method of claim 1, wherein the protein is denatured prior to mass spectrometry.

13. The method of claim 1, wherein the protein is at least 50 amino acids in length.

14. The method of claim 1, wherein the protein is a lysozyme.

15. The method of claim 1, wherein the label is a combination of a hydroxyl radical and at least one different radical.

16. The method of claim 15, wherein the label is combination of a hydroxyl radical and a trifluoromethyl radical.

17. The method of claim 1, wherein the label is a hydroxyl radical.

18. The method of claim 1, wherein the label is generated by X-ray radiolysis.

19. An intact labeled protein produced by the method of claim 8.

20. The intact labeled protein of claim 19, which is an intact labeled lysozyme.
